(19) United States
(12) Patent Application Publication
(10) Pub. No.: US 2010/0299080 A1
(43) Pub. Date: Nov. 25, 2010

(54) DETERMINATION OF OIL DETERIORATION AND CONTROL AND/OR REGULATION OF AN INTERNAL COMBUSTION ENGINE

(75) Inventors: Michael Willmann, Bermatingen (DE); Christian Frey, Friedrichshafen (DE); Gerald Fast, Friedrichshafen (DE)

Correspondence Address: RADER, FISHMAN & GRAUER PLLC, 39533 WOODWARD AVENUE, SUITE 140, BLOOMFIELD HILLS, MI, 48304-0610, US

(73) Assignee: MTU Friedrichshafen GmbH

(21) Appl. No.: 12/781,499

(22) Filed: May 17, 2010

(30) Foreign Application Priority Data
May 18, 2009 (DE) 10 2009 003 200.2

(51) Int. Cl. G01N 33/30

(52) U.S. Cl. 702/25; 73/53.05

(57) ABSTRACT

At least one operating quantity of an internal combustion engine is recorded. A criterion is derived for an oil change from the at least one operating quantity by converting a number of operating quantities, as input quantities of a neural and/or probabilistic computer network, into a number of state quantities characterizing the oil as output quantities of the computer network, wherein at least some of the output quantities are subjected to a check, the criterion being derived from the check.

### CROSS-REFERENCE TO RELATED APPLICATION

This application claims priority to German patent application DE 10 2009 003 200.2, filed on May 18, 2009, which is hereby incorporated by reference in its entirety.

### BACKGROUND

Oil change intervals are usually rigidly stipulated by manufacturers of internal combustion engines, for example, as a function of an operating metric that dictates the oil change, such as the number of operating hours of the engine. Expiration of a predetermined number of engine operating hours is then used as a criterion for an oil change and information is provided to the operator of the internal combustion engine, recommending an oil change. The basis of the oil change intervals determined for this type of method are empirical considerations and/or test bench experiments, by means of which occurring oil deterioration is presumably detected. The actual oil deterioration, on the other hand, occurs according to the actual operation of the internal combustion engine. In such methods as just mentioned, it is critical that a fixed oil change interval cannot be based on the actual state of the internal combustion engine and therefore not on the actual state of the oil.

A first additional measure for improved determination of oil deterioration is known from DE 10 316 315 B4 of the applicant. In it, an internal combustion engine with a crankcase is mentioned, which has openings that can be closed by an inspection hole cover, and with an oil pan to accommodate an oil sump. The status and level of the oil in the oil pan is to be recorded with an oil level sensor, a holder to accommodate the oil status sensor being arranged on the inspection hole cover of the internal combustion engine. The sensor serves to monitor the oil quality, such as, for example, viscosity, contamination, or water content of the oil. Such an essentially good approach, however, is still in need of improvement. It has been found in practice that oil status sensors often do not reach the stability and/or lifetime of an internal combustion engine. In particular, for large engines, like large diesel engines or the like, this solution has proven to be in need of improvement.

A method for evaluation of deterioration of motor oil entrusted to a sensor mechanism is also described from DE 10 048 547 A1. The oil viscosity of the motor oil is measured by a sensor and a temperature sensor is used with the sensor for simultaneous determination of the oil temperature. Oil viscosity and oil temperature in the cooling phase of the engine are thus measured to record a viscosity-temperature characteristic. A viscosity index serves as a criterion indicating that an oil change is desirable. In such a sensor solution, the problems explained above connected with sensors also occur.

An improved solution to determine oil deterioration would be desirable, which can be simply implemented, in particular, and has sufficient, especially improved reliability and/or stability.

Therefore, it would be desirable to provide a method and device with which improved determination of oil deterioration is possible. In particular, the method and device should be implemented comparatively simply and have sufficient reliability and/or stability.

### DETAILED DISCLOSURE OF THE PREFERRED EMBODIMENTS

Herein it is proposed that for derivation of the criterion for deciding when oil should be changed, a number of relevant operating quantities, also referred to as metrics, are converted with a neural and/or probabilistic computer network as input quantities by the computer network to a number of state quantities characterizing the oil as output quantities of the computer network, and at least part of the output quantities are then subjected to a check, in which the criterion is derived from the check.

Empirical considerations and test bench experiments are only partially suited to determine the actual oil deterioration. There are a wide range of variables that influence oil deterioration. Physical-chemical cross-couplings of the variables themselves cannot be recorded even with the latest sensor technology and therefore an empirical approach supplemented by sensors must remain incomplete. However, it is possible to state a significant number of relevant operating quantities for oil deterioration, especially operating quantities of an internal combustion engine and/or its periphery and/or surroundings, from which a reliable criterion for determination of oil deterioration, and therefore for an oil change, can be derived. Such a criterion can be derived with a neural and/or probabilistic computer network. The computer network is used to calculate a number of state quantities that characterize the oil as output quantities of the computer network from the operating quantities provided as input quantities to the computer network. Then, surprisingly, the strength of neural and/or probabilistic computer networks is used to consider generally undefined physical-chemical cross-couplings between the decisive operating quantities. Also proposed herein is a check of at least some of the number of output quantities, the criterion being derived from the check. In a particularly preferred modification of the invention, the check can include a plausibility test and/or a limit value check of the state quantities that characterize the oil.

Overall, even intrinsic parameters, like viscosities or viscosity-temperature characteristics, are only suited under specific conditions to comprehensively indicate oil deterioration. For example, there are additional physical-chemical cross-couplings on the oil that can have opposite effects on viscosity, for example, physical-chemical cross-couplings that increase viscosity, and also reduce viscosity. Even the latest sensor technology of the prior art would therefore document negligible oil deterioration at constant viscosity, although a massive deterioration in quality of the oil is actually present. Such incorrect conclusions can be avoided by deriving a criterion from a number of relevant operating quantities. As explained, relevant operating quantities are preferably operating metric of the internal combustion engine and/or the periphery of the internal combustion engine and/or the surroundings of the internal combustion engine. However, it is also possible to additionally use measured or predicted or otherwise determined state quantities of the oil to determine oil deterioration.

An artificial neural and/or probabilistic computer network is capable of producing the relations between at least the relevant operating quantities and the number of state quantities that characterize the oil and deriving from this a criterion for an oil change by checking at least some of the output quantities. For example, the relevant operating quantities could be measured as input quantities of the computer network or otherwise recorded by engine sensors. The results of the computer network are state quantities characterizing the oil that are relevant as output quantities for oil deterioration.

Output of information concerning an oil change may be a function of the criterion. The information can also be advantageously formed as information concerning the determination method as such or output as additional information concerning the determination method. Because of this, a hardware error or software error or other undefined state of the determination method can be indicated. In addition, a cause for an undefined state of the determination method can be output. The information concerning an oil change can also be information that is neutral with respect to an oil change.

The number of input quantities can advantageously include up to eight input quantities and/or the number of output quantities up to eight output quantities. The number can be configured comparatively flexibly and according to requirements with consideration of mathematical certainty criteria. For example, with respect to certain types of internal combustion engines, specific operating quantities can be used. In addition, the deterioration-relevant state quantities that characterize the oil can be chosen according to the type of oil. If the operating quantities of a type of internal combustion engine or the state quantities of an oil type are constant, they can also be stipulated as constants, in order to optimize computer processing effort. A limitation of the number is not present in practice, since a neural and/or probabilistic computer network can be implemented with acceptable computer power and it is comparatively simple to implement IT technology and hardware even in the context of control and/or regulation for an internal combustion engine.

In one implementation, it is proposed that the number of input quantities have only decisive operating quantities of the internal combustion engine and/or the number of output quantities be only state quantities that characterize the oil. Because of this, a sensor mechanism to measure an oil state can advantageously be almost fully replaced. A number of input quantities in the form of exclusively decisive operating quantities of the internal combustion engine are already furnished during operation of the internal combustion engine. This solution proves to be particularly cost-effective, since the additional IT expense can be implemented comparatively simply in the context of engine control. As initially explained, the input quantities can also be decisive operating quantities of the periphery of the internal combustion engine and/or the surroundings of the internal combustion engine.

The number of decisive operating quantities may include those selected from the group consisting of: oil operating time, oil consumption, engine power, exhaust gas recirculation rate. It has been found that the running time of the motor oil, i.e., oil operating time, has a significant effect on oil quality in the internal combustion engine. An oil consumption usually specific for the internal combustion engine advantageously provides an assertion concerning the fresh oil refilling amount between any oil change intervals. An oil consumption is therefore decisive, on average, for the age of all the oil in the internal combustion engine. Based on different loads of the motor oil in the operating points of an internal combustion engine, an indicator for the released power of the oil is also obtained. Engine power therefore also proves to be a decisive operating quantity for oil deterioration. An exhaust gas recirculation rate also has a distinct, regularly negative effect on oil quality. It was recognized that with increased exhaust gas recirculation rate, an oil should be changed already at an earlier point in comparison with an oil subject to a lower exhaust gas recirculation rate. The reason for this is the increased soot input or the like in the oil.

One implementation uses at least four quantities (namely, oil operating time, oil consumption, engine power, exhaust gas recirculation rate) as input quantities for the computer network. In this context, it was recognized that, in each case, these four operating quantities are particularly suited for reliable prediction of oil deterioration.

If necessary, the input quantity of oil consumption, if it is known, can optionally be omitted in a computer network with respect to a special type of internal combustion engine, as a constant of the internal combustion engine. In this case, the modification of a method configured in this way would be specific for a specific type of internal combustion engine. A determination method of oil consumption, however, advantageously contains the oil consumption as input quantity for the computer network, in order to be usable for different types of internal combustion engines.

Overall, a sufficient number of characterizing state quantities of the oil can be determined as output quantities advantageously by means of the aforementioned number of decisive, or relevant, operating quantities. Depending on the requirements, a relevant number of state quantities that characterize the oil can be established.

In one implementation, the number of input quantities also includes a number of characterizing state quantities of the oil. These can prove to be advantageous, if a characterizing state quantity of the oil can be measured comparatively simply and/or cost-effectively. This especially concerns the state quantity of viscosity of the oil or a viscosity index from the viscosity-temperature diagram of the oil. The stipulation of a specific oil quality is also possible. Viscosity sensors or other sensors can be simply integrated in the present method.

In one implementation, the number of characterizing state quantities of the oil includes those selected from the group consisting of: viscosity, temperature, oxidation, nitration, soot content. In one context, all four state quantities in particular (namely viscosity, oxidation, nitration and soot content of the oil) can be determined in the context of the neural and/or probabilistic artificial computer network. In another context, the viscosity can be omitted as output quantity, since it is available as input quantity.

It is proposed, in particular, to provide checking of the number of output quantities with a plausibility test for the state quantities that characterize the oil. A plausibility check has the particular advantage that impossible results or otherwise inconsistent results can be avoided in the output quantities of the artificial computer network. In particular, a plausibility check can include a test whether a running time of the motor oil lies within the operating time of the internal combustion engine. A plausibility check can also include a test whether a specific oil consumption lies within the total amount of oil. A plausibility check can also include the test whether an engine power lies below the maximum power of the internal combustion engine. A plausibility check can also include the test whether an exhaust gas recirculation rate lies below the maximum possible exhaust gas rate. These and other plausibility checks can be integrated comparatively simply by comparison devices, like compensators or similar logic components in a control.

The check of the number of output quantities also advantageously includes a limit value check of the state quantities characterizing the oil. A threshold value can be stated for each characterizing state quantity of the oil, which represents a threshold between a first range of the state quantity, which still indicates sufficient quality of the oil, and another second range that indicates a no-longer-sufficient quality of the oil concerning the state quantity. A threshold value for each state quantity characterizing the oil can be used in the context of a limit value check as the limit value. As in the plausibility check, the limit value check, in particular, can include at least some of the output quantities, but especially all of the output quantities. The check can be a separate and/or summary check of the number of output quantities.

The check can be laid out as required, for example, in the context of a separate check, already on surpassing a single limit value of an output quantity, a criterion for an oil change can be derived that recommends an oil change. In addition or as an alternative, a summary check of the number of output quantities can also be conducted, in which a criterion for an oil change with recommendation of an oil change is only issued if the plurality of threshold values of the output quantities is surpassed. A summary check of the number of output quantities can also include weighing process steps designed to weigh the degree of surpassing and/or falling short of the threshold value for the number of output quantities relative to each other. Overall, in the context of application-specific embodiments of this modification, sensitivity of the determination method can be adjusted by setting the sensitivity of the control step.

In particular, the criterion can be formed as a parameter with a limit, in which, in the case of surpassing of the limit by the parameter, information concerning the oil change includes a recommendation for an oil change. The recommendation can also contain a timeframe, within which the oil change is recommended.

With respect to calculation by the artificial network (in the context of a comparatively simple variant of the computer network), a mutually independent calculation for each of the output quantities can be carried out by the neural and/or probabilistic computer network. In such a calculation, the result concerning a first state quantity would have no effect on the result concerning a second state quantity of the oil. In the context of a more demanding modification, likewise provided with higher reliability, a mutually related calculation can be conducted for each of the output quantities by the neural and/or probabilistic computer network. The result concerning a first state quantity would then also have an effect on the result concerning a second state quantity. Such modifications can be implemented, for example, in the context of a recursive inclusion of the computer network in a loop or in the context of deterministic, coupled computer procedures.

In principle, the artificial computer network, as required, can advantageously be designed as a neural and/or probabilistic computer network. A one- or multilayer perceptron network has proven to be particularly suited. A single-layer perceptron network has comparatively limited computer and IT costs. A multilayer perceptron network with at least one concealed plane of neurons is more reliable. A radial base function network is also advantageous. A radial base function network can be laid out application-specific, in particular. A network according to adaptive resonance theory (ART) or predictive, adaptive resonance theory (ARTMAP) has proven to be especially flexibly and simply adaptable to different operating states. These have the advantage that with appropriate training, they are capable of being flexibly adjusted to different input quantities as a result of different decisive operating quantities. A Bayesian network has proven to be particularly preferred for a probabilistic computer network.

Concerning the device, an electrical device for control and/or regulation of an internal combustion engine of the type mentioned in the introduction may be used, in which it is proposed that computer devices are provided to derive the criterion, having a neural and/or probabilistic computer network with inputs for input quantities in the form of a number of relevant operating quantities and designed to convert the input quantities to a number of state quantities characterizing the oil as output quantities, and containing comparison and logic units designed to subject the number of output quantities to a check and deriving the criterion from the check of all oil output quantities. The computer device includes, in particular, a processor, which is laid out to perform calculations with a neural and/or probabilistic computer network. The processor or optionally an additional processor is laid out to perform a check of the output quantities. For this purpose, a computer device in the form of a comparator or the like can also be used.

Concepts disclosed herein may be implemented as a computer program product. An electrical device of the aforementioned type may be used to control and/or regulate an internal combustion engine. Preferably, an internal combustion engine additionally has one or more sensor(s) or recording devices to record at least one of the operating quantities indicative of oil deterioration and/or at least one characterizing state quantity of the oil.

Practical examples are now described below with reference to the drawings. The drawings do not necessarily represent the practical examples to scale, but instead, where it is useful for explanation, are schematized and/or slightly distorted. With respect to additions of instructions directly recognizable from the drawing, the pertinent prior art is referred to. It must then be considered that numerous modifications and changes concerning the form and details of a variant can be made without deviating from the general idea of the invention. The features of the invention disclosed in the description, in the drawing and in the claims can be essential both individually and in any combination for the modification of the invention. In addition, all combinations of at least two features disclosed in the description, drawing and/or claims fall within the scope of the invention. The general idea of the invention is not restricted to the exact form or details of the preferred variant depicted and described below or restricted to an object that would be restricted in comparison to the object claimed in the claims. In the stated measurement ranges, values also lying within the mentioned limits are disclosed as limit values and are arbitrarily usable and claimed.

Additional advantages, features and details of the invention are apparent from the following description of preferred practical examples with reference to the drawing. In particular, in the drawing:

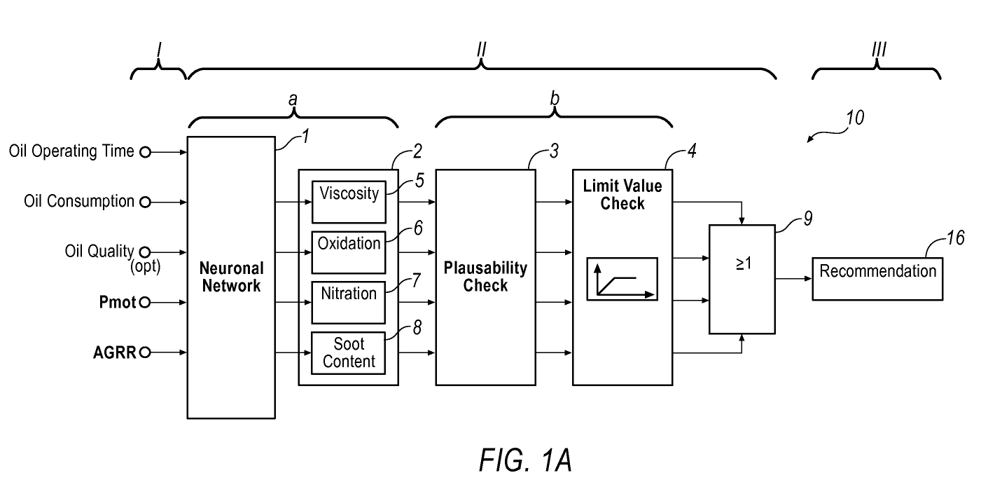

FIG. 1A: shows a schematic view to explain a method for determination of oil deterioration and the electrical device for control and/or regulation of an internal combustion engine;

FIG. 1B: shows a schematic view to explain a method for determination of oil deterioration and the electrical device for control and/or regulation of internal combustion engines;

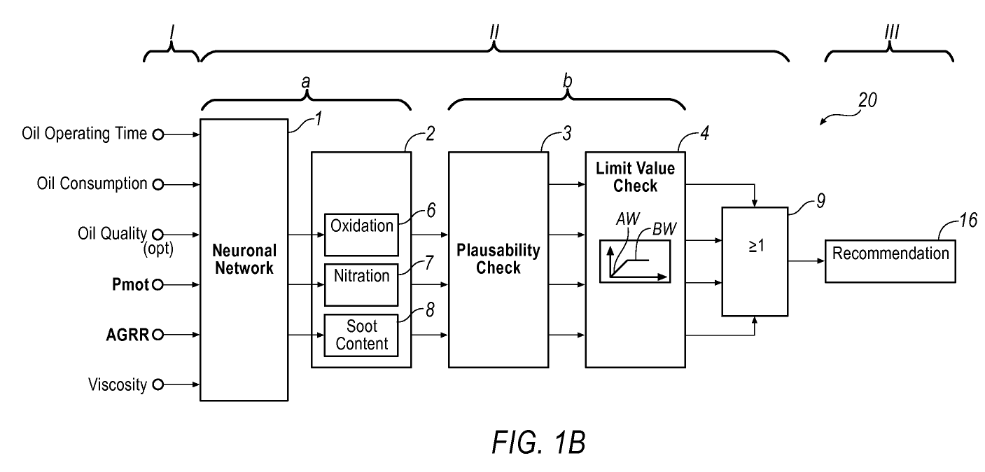

FIG. 2: shows an example of a neural artificial network, as stated in FIG. 1A and FIG. 1B (in the present case, a multilayer perceptron network with a concealed neuron layer), for determination of oxidation as a state quantity characterizing the oil;

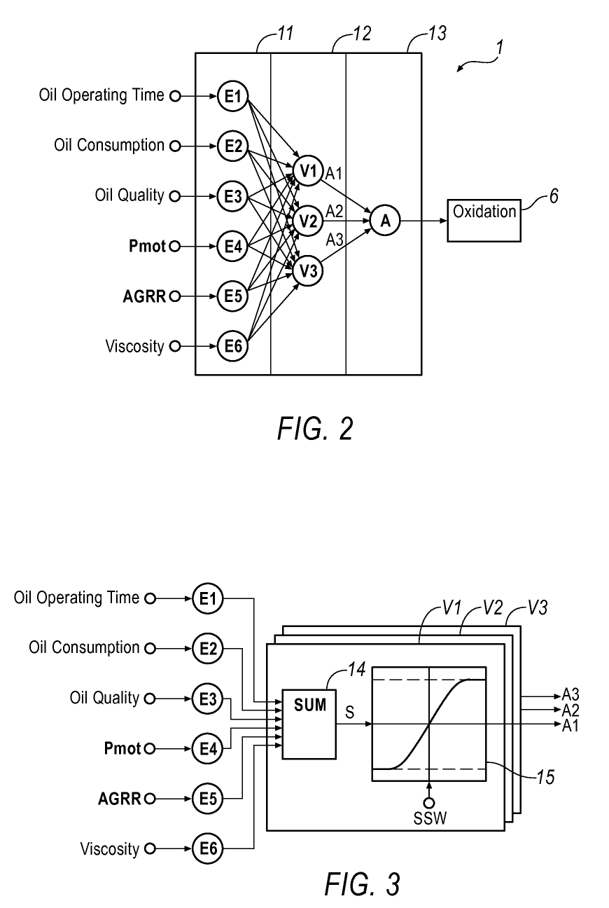

FIG. 3: shows a block circuit diagram of the concealed neuron layer in the network of FIG. 2;

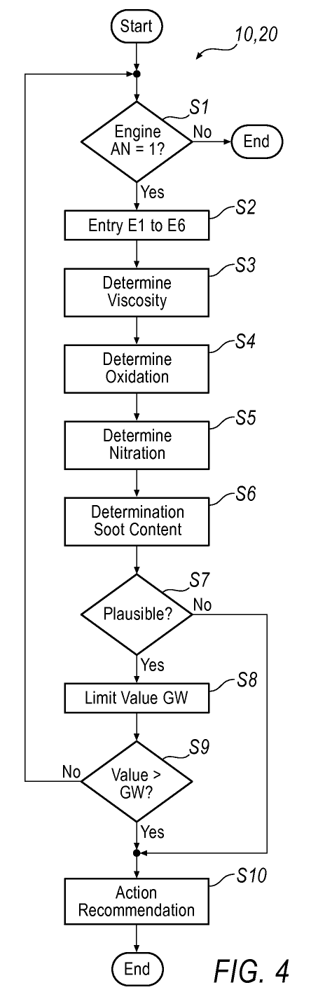

FIG. 4: shows a flow chart for a method of function of an electrical device for execution of the method and/or a computer program product.

A method 10 for determination of oil deterioration in an internal combustion engine (not further shown) is shown in the context of a block diagram to depict a first version in FIG. 1A. A second method 20 is shown in FIG. 1B. For identical or similar elements or elements with identical or similar function, the same reference numbers are used here in the interest of simplicity.

Method 10, 20 initially proposes recording I of at least one operating quantity relevant for oil deterioration and then derivation II of a criterion for an oil change from the at least one relevant operating quantity and finally output III of information concerning an oil change as a function of the criterion. According to the concept of the invention, for derivation II of the criterion, two method steps are conducted. In a first step (a), a number of decisive operating quantities are converted as input quantities of a neural artificial computer network 1 by the computer network 1 into a number of state quantities characterizing the oil as output quantities of the computer network 1. In a second step (b), at least some of the output quantities are subjected to a check, in this case a plausibility check 3 and a limit value check 4, in which the criterion 9, here in the form of a parameter with limit, is derived from the check. Information is output here in the form of a recommendation 16 as a function of criterion 9.

In the method 10, five input quantities are used, in order to convert them with the neural network 1 to four output quantities. The neural network here is formed as a two-layer perceptron network, which is further explained with reference to FIG. 2 and FIG. 3 with input quantities E1 to E6 and output quantities A. The input quantities are formed in method 10 exclusively by five decisive operating parameters of the internal combustion engine. The output quantities are exclusively the state quantities characterizing the oil. Specifically, the output quantities for the oil operating time ÖL(h), oil consumption ÖL(Vb), oil quality ÖL(Q), motor power Pmot, and exhaust gas recirculation rate AGRR serve as input quantities. As output quantities, the viscosity 5 and oxidation 6 and nitration 7 of the oil, and also the soot content 8 of the oil, are determined by a calculation 2. The oil quality ÖL(Q) is optional here to the extent that it can be omitted and the output quantities can be fully calculated as depicted. Likewise, in a variant not shown here, the oil consumption ÖL(Vb) can drop out as input quantity for a case, in which this is constant for the use range of the method. This is the case, for example, if the method is used only for internal combustion engines of a specific type.

All the aforementioned operating quantities are quantities that are ordinarily established in the context of an electrical device for control and/or regulation of an internal combustion engine, for example, an engine control—the aforementioned operating quantities are therefore already available as part of ordinary engine control. It is also to be understood that the aforementioned operating quantities, as well as other input quantities not mentioned here, are present in other practical examples in the form of a significant characteristic. Such a characteristic has the advantage that it can, for example, include an averaged weighting of the relevant operating quantity over an appropriate observation period of the operating quantity in the context of engine control. For example, a characteristic that characterizes the average engine power can be used for engine power and this optionally with the characteristic for maximum engine power. In principle, it is also conceivable that the relevant operating quantity is available in the form of a dataset, a map or a data matrix. The characteristic can also contain the time trend of the relevant operating quantity over the observation period. It is understood that in this case, conversion of the characteristic or map or similar dataset for the relevant operating quantity occurs into a form of input quantity appropriate for the neural network 1. Additional information of a time trend of the characteristic or the like can be considered in formulating the input quantity.

In method 10 according to the first practice example, the neural network 1 is formed from four individual neural partial networks for determination of the four state quantities characterizing the oil as output quantities. The type of partial network is explained with reference to FIG. 2 and FIG. 3.

A method 20 with generally the same design as method 10 is shown in FIG. 1B, which, however, differs in the choice of input quantities and output quantities. In method 20, in addition to the relevant operating quantities mentioned in method 10, a state quantity characterizing the oil is also used as input quantity. The viscosity is used here as the state quantity characterizing the oil Visk, in order to additionally use the relevant operating quantity as additional input quantities in neural network 1. The viscosity Visk is determined for this purpose by means of a comparatively expensive sensor, or a characteristic representing the viscosity. The method 20 therefore uses a total of six input quantities to derive criterion 9, five of which are the operating quantities of the internal combustion engine or its periphery, and one of which is a state quantity characterizing the oil. With respect to the design of method 20, this has the advantage that only three output quantities are to be determined as state quantities characterizing the oil, namely, oxidation 6, nitration 7 and soot content 8. The additional characterizing state quantity viscosity Visk is already available for determination of oil deterioration as a result of measurement. This has the advantage that the neural network 1 consists of only three individual neural partial networks, which, however, each have six input quantities. The partial networks of the neural network 1 of method 10 have only five input quantities. The neural network of method 20, to this extent, is overdefined to a higher degree than the neural network 1 of method 10. The neural network 1 of method 20 therefore has the advantage that it should arrive at more reliable results, and also, if necessary, should get by with lower computer expense or time expenditure.

This can mean that a check (b) in the context of the step for derivation (II) of a criterion 9 is simpler and less prone to error than in a method 10 according to the first variant. However, in method 20 there is higher measurement expense on the input side. Both methods 10, 20 can be selected as required and are expedient for an appropriate use.

The design of the step for the check (b) in method 10, 20 is also essentially the same. In a first step of a plausibility check 3, the output quantities of the neural network 1, i.e., oxidation 6, nitration 7 and soot content 8 in method 20 and viscosity 5 in addition in method 10, are checked to see if they lie in a suitable range. For this purpose, logically reasonable assumptions concerning the stipulated limits of the output quantities are used. Specifically, for each of the output quantities, a plausible range is stipulated. If the output quantity determined by the neural network 1 does not fall in the plausible range, method 10, 20 can either be interrupted and repeated or interrupted and information concerning the determination method 10, 20 output instead of the recommendation 16. For example, this can be information concerning a hardware error or other information concerning the non-plausible result of the output quantity.

For each of the output quantities, namely, for oxidation 6, nitration 7 and soot content 8 in method 20 and viscosity 5 in addition in method 10, a separate limit value check 4 is then conducted. This means for each of the mentioned output quantities, it is checked whether this is still admissible or not with respect to a threshold value of the characterizing state quantity maximally admissible for oil quality. In the limit value check 4, threshold value SW is symbolically shown. If a state quantity characterizing the oil determined by the neural network 1 assumes an acceptable value AW, the limit value check 4 for this state quantity can yield a value number that is suitable for forming the criterion 9 in the subsequent step. In the present case, the value number “zero” is assigned to a state quantity as output quantity of the neural network 1, if it assumes an acceptable value AW, and the value number “one” is assigned, if it surpasses a threshold value SW. In the present case in method 10, 20, this means that during summation of all value numbers for the state quantities as output quantities of the neural network 1, only a sum below 1 is formed, if all state quantities assume an acceptable value AW. In other words, this type of limit value check 4 means that in the case, in which only a single state quantity surpasses a threshold value SW, the criterion 9 surpasses the value “one”. The criterion 9 is formed here as a parameter with limit “one”, in which the parameter is the sum of the value numbers for the state quantities 5, 6, 7, 8 and the limit is 1. As a result, a method 10, 20 of this type leads to a situation, in which an oil change is recommended in the context of information, if only one single state quantity, viscosity 5, oxidation 6, nitration 7 or soot content 8, surpasses a threshold value SSW.

Other parameters with a limit can also be used to form a criterion. For example, a softer weighting of the output quantities can be conducted, so that information with recommendation 16 for an oil change is only conducted, if all state quantities determined by the neural network 1 as output quantities surpass a threshold value. In this case, the value number of an output quantity would be established at 0.25 if the threshold value is surpassed and the value number established at 0 if the state quantity has an acceptable value AW. Sum formation then leads to the parameter value 1 only in the case, in which all output quantities surpass a threshold value SW, which, in this case, corresponds to the limit and leads to output of a recommendation for an oil change.

FIG. 2 shows a two-layer perceptron network for formation of a partial network—here for formation of the partial network for determination of oxidation 6—for the neural network 1 in a method 10, 20. The partial network depicted in FIG. 2 has an input layer 11 to record and normalize the input signals E1 to E6. The viscosity Visk is also shown here as possible input value E6 as part of method 20. In the case of method 10, the viscosity Visk drops out as input value E6. The input layer 11 of the partial network of FIG. 2 for method 10 would therefore have only input values E1 to E5, as explained above. The second layer of the partial network in FIG. 2 is a concealed layer 12 with artificial neurons V1 to V3 for determination of output quantities A1, A2 and A3. From the output values A1 to A3, the oxidation 6 is determined in the output layer 13 as output quantity A. In the case of method 10, another three partial networks of this type are used to determine viscosity 5, nitration 7 and soot content 8 in the neural network 1. In the case of method 20, another two partial networks are implemented to determine nitration 7 and soot content 8 in the neural network 1.

For further explanation, the oxidation 6 can be determined by means of the following equation for output value A:

A=c₀+c₁(E₁)+c₂(E₂)+c₃(E₃)+c₄(E₄)+c₅(E₅)+F,
in which
F=c₆ (E₆), if a viscosity sensor is present, as in method 20, or
F=0, if no viscosity sensor is present, as in method 10.
   The present example shows that a method 10 and a method 20 can also be offered as selectable alternatives in the context of a method for determination of oil deterioration. In other words, a method for determination of oil deterioration can contain a decision step, which checks the option of availability of a viscosity determination. If a viscosity determination is available (F=C₆ (E₆)), a method 20 according to the second variant is available to the decision step. If a viscosity determination is not available (F=0), a method 10 according to the first variant is available for the decision step.

FIG. 3 shows as block diagram for explanation of additional details a concealed layer 12 of a neural partial network, in this case with the first, second and third artificial neurons V1, V2, V3 of the partial network in FIG. 2. Each artificial neuron includes a sum formation 14 of the input quantities E1, E2, E3, E4, E5, E6 and an activation function 15, which can also be referred to as threshold value function. Sum formation yields the sum value S as value. It is to be understood that the sum formation 14 is also a weighted summation over the subscript j with weights Wᵢⱼ, in which the subscript i is the corresponding weight for the i-th neuron Vᵢ.

The abscissa of the threshold value function or activation function 15 is represented by the sum value S. The ordinate of the activation function 15 is a value A1 for neuron V1 or a value A2 for neuron V2 or a value A3 for neuron V3, which is dependent on the choice of activation function. In the present case, a sigmoid tangent function is chosen as activation function. The neuron Vᵢ (i=1, 2, 3) only fires when the value of sum S is greater than a threshold value SSW of the activation function. Sum values below the threshold value SSW of the activation function are suppressed.

FIG. 4 shows a program flow chart for a computer program underlying methods 10, 20. This has the following steps.

After beginning the method START, it is checked initially in a first step S1 whether the engine is running. If a characteristic AN prescribed for this is at 1, in a subsequent step S2, entry of the input quantities E1 to E5 in the case of method 10 or input quantities E1 to E6 in the case of method 20 occurs.

The subsequent steps S3, S4, S5, S6 for method 10 or steps S4, S5, S6 for method 20 represent determination of the already described state quantities characterizing the oil as output quantities of the neural network 1. For example, in step S4 a state quantity characterizing the oil in the form of oxidation is determined as the output quantity of the neural network 1, specifically with a partial network as shown in FIG. 2 and FIG. 3. The soot content is similarly determined in step S6.

If the engine was operated, for example, over a longer operating time with a higher exhaust gas recirculation rate (AGRR—input quantity E5), the input factor for the input quantity AGRR or E5 rises.

For the state quantities of viscosity, oxidation and nitration, there are other principles that are not explained in detail here. Finally, these are chemical-physical cross-couplings that, after training of the neural network 1, are recorded by it. It is merely mentioned as an example that an engine stress measured by the engine stress through input quantity E4 (Pmot) and the fresh oil quality (input quantity E3, ÖL(Q)) can reduce the viscosity of the motor oil. The other input quantities E1, E2 and E5 should ordinarily increase the viscosity of the motor oil. In particular, it is assumed that with increased nitration, oxidation and soot content, the viscosity of the oil is increased.

The latter can lead to a case, in which, at constant viscosity (reduction of the viscosity by mechanical shear and fresh oil quality, on the one hand, and an increase in viscosity by nitration, oxidation and soot content, on the other hand), a massive quality deterioration of the motor oil has occurred. In the present case of the method, each of the output quantities determined in method steps S3, S4, S5, S6 is subjected to a plausibility check for soot content, for example, in method step S7. With a positive plausibility check, each of the output quantities is subjected to a limit value check in method step S8. The result of check S7, S8, both for soot content and for nitration, oxidation and viscosity, is now used to form a criterion in the form of a parameter with a limit in method step S9. The parameter is referred to as “value”, the limit is designated as “GW”. For the case in which the parameter lies above the limit, in a subsequent method step S10, an oil change recommendation is sent to the operator of the internal combustion engine. For example, if the calculated soot concentration of the motor oil lies outside the stipulated limit value, this can contribute to issuing of a positive oil change recommendation. The latter is dependent on the formation of the criterion in step 9 in FIG. 1A and FIG. 1B. It is also dependent on whether a calculated soot content of the motor oil leads to an oil change recommendation within the prescribed limit value. If the additional output quantities viscosity, oxidation and nitration lie above the limit value, despite an acceptable soot content, an oil change recommendation could be output.

If no oil change recommendation is output, the method in step S10 proposes that no action recommendation is output and the process repeats after a certain time interval.

Overall, based on the methods 10, 20 proposed here, significantly reduced operating costs may result for the operator of internal combustion engine. This is due to the fact, among other things, that the oil change intervals are optimized, i.e., occur as required and not arbitrarily. Concepts disclosed herein are almost cost-neutral in an engine control as a software-based solution. In addition, such concepts permit a prediction concerning when an oil change will be required in a certain type of operation. The time stipulation for the operator of an internal combustion engine is therefore reliable.

In addition, a method can be trained and further developed based on implementation of a neural and/or probabilistic artificial computer network. For example, reference measurements in the oil laboratory or other experience values can be used for continuing training of the neural network and/or the probabilistic network, in order to improve the information content of the method proposed here.

1-18. (canceled)

19. A method, comprising:
recording at least one operating quantity of an internal combustion engine; and
deriving a criterion for an oil change from the at least one operating quantity by converting a number of operating quantities, as input quantities of a neural and/or probabilistic computer network, into a number of state quantities characterizing the oil as output quantities of the computer network, wherein at least some of the output quantities are subjected to a check, the criterion being derived from the check.

20. The method of claim 1, further comprising providing output concerning an oil change as a function of the criterion.

21. The method of claim 1, wherein the number of input quantities includes up to eight input quantities and/or the number of output quantities includes up to eight output quantities.

22. The method of claim 1, wherein the input quantities include all relevant operating quantities of the internal combustion engine and/or the output quantities include all state quantities characterizing the oil.

23. The method of claim 1, wherein the operating quantities includes quantities selected from the group consisting of: oil operating time, oil consumption, engine power, and exhaust gas recirculation rate.

24. The method of claim 1, wherein the input quantities include characterizing state quantities of the oil, including at least one of viscosity and oil quality.

25. The method of claim 1, wherein the number of characterizing state quantities of the oil includes quantities chosen from the group consisting of: viscosity, temperature, oxidation, nitration, soot content, and oil quality.

26. The method of claim 1, wherein checking of the output quantities includes a plausibility check of the state quantities.

27. The method of claim 1, wherein checking of the number of output quantities includes a limit value check of the state quantities characterizing the oil.

28. The method of claim 1, wherein the check includes at least one of a separate and a summary check of the output quantities.

29. The method of claim 1, wherein, the computer network, for each of the output quantities, performs an independent calculation.

30. The method of claim 1, wherein, the computer network, for each of the output quantities, performs a dependent calculation.

31. The method of claim 1, wherein the criterion is a parameter with a limit, wherein in the case of surpassing of the limit by the parameter, information concerning the oil change includes a recommendation for an oil change.

32. The method of claim 31, wherein the recommendation includes a timeframe within which an oil change is recommended.

33. The method of claim 1, wherein the computer network is chosen from the group of consisting of: a single- or multilayer perceptron network, a radial base function network, a network according to adaptive resonance theory (ART) or predictive adaptive resonance theory (ARTMAP), and a Bayesian network.

34. A system, comprising:
at least one sensor configured to record at least one operating quantity relevant to oil deterioration;
a computing device configured to derive a criterion for an oil change from the at least one operating quantity, the computing device including a neuronal and/or probabilistic computer network with inputs for input quantities in the form of operating quantities, the network being configured to convert the input quantities to state quantities characterizing the oil as output quantities, the network including contains comparison and logic units designed to subject the output quantities to a check and to derive the criterion from the checking of all output quantities.

35. The system of claim 34, further comprising an output device to output information concerning an oil change as a function of a criterion.

36. The system of claim 35, further comprising an internal combustion engine, having an electrical device for control and/or regulation of the internal combustion engine according to the information.

37. A computer program product for storage in a medium by a computer, and readable by the computer unit, having a software code section that initiates a processor in the computer to execute the method according to claim 1.
